(19) United States
(12) Patent Application Publication
(10) Pub. No.: US 2012/0003722 A1
(43) Pub. Date: Jan. 5, 2012

(54) CARBON DIOXIDE REMOVAL SYSTEMS

(75) Inventors: Robert B. Polak, New York, NY (US); Meyer Steinberg, Melville, NY (US)

(73) Assignee: COAWAY LLC, Crozet, VA (US)

(21) Appl. No.: 13/256,831

(22) PCT Filed: Mar. 18, 2010
(86) PCT No.: PCT/US10/27761

§ 371 Date: Sep. 15, 2011

Related U.S. Application Data
(60) Provisional application No. 61/161,122, filed on Mar. 18, 2009.
(60) Provisional application No. 61/262,951, filed on Nov. 20, 2009.
(60) Provisional application No. 61/289,498, filed on Dec. 23, 2009.

(51) Int. Cl. C12N 1/12; E21B 43/00; A62B 7/08; C12M 1/00; B01D 53/14; A23L 2/54

(52) U.S. Cl. 435/257.1; 96/218; 96/205; 95/180; 426/477; 422/120; 435/283.1; 166/369

(57) ABSTRACT

A system for removing CO₂ from air such as atmospheric air is described that uses a cooling tower, a pseudo-cooling tower, or a wind capture device to provide a large volume of atmospheric air. A CO₂ capture apparatus is positioned to contact atmospheric air moving towards or within the cooling tower, pseudo-tower, or wind capture device, and includes wherein the CO₂ capture apparatus includes a CO₂ binding agent that binds to CO₂ in atmospheric air. An associated reprocessing apparatus releases C02 from the binding agent, directs the released CO₂ to a CO₂ storage chamber, and returns the binding agent to the CO₂ capture apparatus. A system for removing CO₂ from flue gas is also described.

This application claims the benefit of PCT/US2010/027761, filed Mar. 18, 2010, U.S. Provisional Application Ser. No. 61/161,122, filed Mar. 18, 2009, U.S. Provisional Application Ser. No. 61/262,951, filed Nov. 20, 2009, and U.S. Provisional Application Ser. No. 61/289,498, filed Dec. 23, 2009, all of which are incorporated by reference herein.

### BACKGROUND

It is now well known that during the last 150 years carbon-dioxide (CO₂) concentration in the earth's atmosphere has increased by nearly 35% from approximately 280 to 385 parts per million (ppm), which today amounts to a total of about 3000 gigatonnes. Moreover, global annual emission of CO₂ currently amounts to about 26 gigatonnes per year, of which 13 gigatonnes winds-up in the atmosphere (the rest being absorbed by the oceans) which is estimated to increase atmospheric CO₂ by about 2 ppm/yr.

Because of the rising level of these so called Green House Gasses (“GHG”) a great deal of discussion, concern and development efforts have been directed to reducing, halting or even reversing the growth of carbon emission. Many approaches have been suggested, including more efficient use of energy, a shift away from fossil fuels to renewable fuels, moving towards a hydrogen economy, building more nuclear power plants, etc. However, while some or all of these initiatives may play a significant role in our energy future, it is doubtful that the world will give up or even materially reduce its use of fossil fuels (i.e., coal, petroleum natural gas) in the foreseeable future.

Thus a significant amount of effort has been directed towards the development of processes which could capture, sequester and/or reuse carbon that would otherwise be vented into the atmosphere. In the future, pursuing strategies to lower the “carbon footprint” of various operations may become essential for various industries and companies as laws are passed imposing a tax on carbon emission. Another idea, which may gain traction if deemed to be economically viable, would be to capture CO₂ from the atmosphere. This might create operations that would have a zero or even a negative “carbon footprint”.

How to solve this looming problem at acceptable cost has been under discussion for some time. Many potential solutions have been considered but none are entirely satisfactory. Thus, most of the players have reluctantly concluded that no silver bullet exists so we will have to be satisfied with a range of partial “good but imperfect” solutions.

Since fossil (especially coal) fueled electrical power plants generate a large part of the world's CO₂, a great deal of attention has been focused on how these point sources of greenhouse gases could be controlled or even eliminated. In theory it should be possible to recover the CO₂ from stack gases and permanently sequester them underground. However, it appears that this will be very expensive, and whether buried CO₂ will remain permanently underground is unknown.

There are many possibilities for reducing CO₂ emissions such as the use of nuclear power or renewable energy sources. Unfortunately, nuclear power would require a huge capital investment and for this reason as well as its associated “fear factor” would make a massive switch to nuclear power very highly unlikely. On the other hand, renewable energy sources (i.e., wind, solar, hydro, etc), have cost, capacity limitations, and other impediments that will make it very hard for these niche players to significantly impact the dominance of fossil-based electrical generation.

With this in mind, some have suggested removing CO₂ that is already in the air may be the answer. This sounds attractive in theory because it would remove CO₂ that was previously vented without having to change current practices. However, removing CO₂, which is a very dilute gas in the atmosphere, is a technically demanding problem.

For example, Klaus Lackner has proposed capturing CO₂ from the atmosphere using “synthetic trees.” These include a fabric screen bearing an absorbent coating such as limewater, supported by a goal post-shaped structure, that capture CO₂ in the atmosphere that is brought to the structure by the wind. The absorbent coating can then be washed off and stored or recycled. Other CO₂ capture systems have also been described by Lackner in U.S. Patent Publication Nos. 2006/0051274, 2006/0186562, and 2008/0031801.

Another example is provided by U.S. Pat. No. 4,197,421, in which Meyer Steinberg suggested that atmospheric CO₂ could be captured by scrubbing it from the air and reacting it with aqueous sodium hydroxide (NaOH) so as to make an aqueous solution of sodium carbonate/bicarbonate. These salts as well as additional water would then be decomposed by electrolysis in a three compartment electrolytic cell to produce hydrogen gas (H₂) and CO₂ while at the same time regenerating the NaOH which would then be recycled so it could capture more CO₂. The CO₂ and H₂ would then be thermocatalytically combined to produce methanol that could be used to make synthetic carbonaceous fuels (such as gasoline) and intermediate chemical feedstock for the production of other chemicals. It appears that the oxygen generated at the anode would be vented into the atmosphere.

Unfortunately, this approach is prohibitively expensive. To capture and regenerate the CO₂ and produce the needed H₂ by electrolyzing water would require a huge amount of electricity. Indeed, it seems that this process would require so much electricity that dedicated power plant(s) would have to be built for the electrolysis aspect alone, make it an extremely expensive, capital intensive undertaking. For a similar approach, see U.S. Pat. No. 7,605,293 by Olah et al.

More recently Drs. F. Jeffrey Martin and William L. Kubic Jr. of Los Alamos National Laboratory presented a variant of this concept in far more detail at the Feb. 2, 2008 2nd Annual Alternative Energy NOW Conference. The estimated cost for gasoline prepared by the “Los Alamos process” was projected to be about $4.50 to $5/gallon. Moreover, to produce 17,000 to 18,000 Bbl/day of gasoline would require an investment of at least 5.2 billion dollars, of which 3 billion dollars would go to build two 1-gigawatt (GW) nuclear power plants. However, based on its history and current state of the nuclear industry, these cost projections appear to be much too low.

According to the Los Alamos study, in order to produce this much gasoline, about 7,800 tonnes/day of CO₂ would have to be recovered from the air, which would require about 450,000,000 normal cubic meters (Nm³)/hr of air be processed. This is the amount of air that would normally flow through the 6 cooling towers required to operate the facility.

However, to put this process in a broader context, consider what it would take to remove enough CO₂ from the atmosphere to be considered for the Virgin Earth Challenge. This challenge offers a $25 million prize to anyone who can provide a system to remove an amount of greenhouse gases from the atmosphere equivalent to 1 billion tonnes of CO₂ (about 0.03% of the CO₂ in the atmosphere) each year for at least a decade. To remove enough CO₂ from the atmosphere to qualify for this prize would require the construction of about 360 “Los Alamos sized” nuclear plants which would cost at least $1.9 trillion and require mobilizing an industrial infrastructure on a massive scale to build seven times the number of nuclear plants that now exist in the US.

There are at least two reasons why it is difficult to remove CO₂ from the air. First, the CO₂ is so dilute that a huge amount of air has to be moved to process sufficient air to remove a significant amount of CO₂, which would typically take large facilities and a lot of energy. Second is the high amount of energy that must be used by conventional methods to capture and release CO₂, which is especially costly when it is used to handle material that is very dilute.

There are two conventional methods to capture and subsequent release CO₂. One is to capture CO₂ with an aqueous amine solution; the other is to separate it from the air by passing it through a selective membrane. Unfortunately both of these processes consume a lot of energy.

Normally the conventional absorption process is used to remove relatively concentrated CO₂ from mixtures such as flue gas or contaminated natural gas, by reacting it with aqueous amine (usually an alkanolamine) solution in accordance with the following reaction:

C₂H₄OHNH₂+CO₂+H₂O→C₂H₄OHNH₃⁺+HCO₃ [Eq 1]

Once the CO₂ has been removed from the gas stream, the remaining gas can move on to its intended use, while the remaining saturated amine solution is made ready for regeneration. In this step the amine solution can be regenerated by driving off CO₂ in specialized equipment such as flash tanks and/or stripper columns operated at high temperature or low pressure. However, heating up the solution requires a lot of energy to recover a very small amount of CO₂, and therefore the energy needed per unit of recovered CO₂ is very high.

The details regarding membrane separation are quite different, but the result in terms of energy usage is essentially the same. Typically membrane devices for gas or vapor separation operate under continuous steady-state conditions with three streams. The feed stream (a high-pressure gas mixture) passes along one side of the membrane. The molecules that permeate the membrane are swept out by gas on the other side of the membrane in the so called “permeate stream.” The remaining non-permeating molecules that remain on the feed-stream side exits as the “retentate stream.” The pressure difference across the membrane drives the permeation process. Each component in the feed mixture has different characteristic permeation rate through the membrane and this difference is what permits the desired separation. Unfortunately, selectivity is not complete. Carbon dioxide passes, but so does some of the remaining air, and therefore the resultant CO₂ isn't completely purified in a single pass. To get a nearly pure CO₂ “permeate stream” requires many passes under almost any circumstances. However, since this process starts with a very low concentration (<400 ppmv) of CO₂ in air, it will take an extraordinary number of passes, making it very energy intensive and expensive.

Accordingly, what is needed is a method for removing CO₂ from air with lower energy requirements, which could therefore be carried out far less expensively.

### SUMMARY OF THE INVENTION

In one aspect, the present invention provides a system for removing carbon dioxide (CO₂) from atmospheric air that includes a cooling tower, a pseudo-cooling tower, or a wind capture device. The system also includes a CO₂ capture apparatus positioned to contact atmospheric air moving towards or within the cooling tower, pseudo-tower, or wind capture device, as well as a reprocessing apparatus in communication with the CO₂ capture apparatus. The CO₂ capture apparatus includes a CO₂ binding agent that binds to CO₂ in atmospheric air, and the reprocessing apparatus releases CO₂ from the binding agent, directs the released CO₂ to a CO₂ storage chamber, and returns the binding agent to the CO₂ capture apparatus.

In another aspect, the invention provides a system for removing carbon dioxide from flue gas that includes a CO₂ capture apparatus positioned to contact flue gas moving from or within a smokestack and a reprocessing apparatus in communication with the CO₂ capture apparatus. The CO₂ capture apparatus includes a CO₂ binding agent that binds to CO₂ in atmospheric air, and the reprocessing apparatus that releases CO₂ from the binding agent, directs the released CO₂ to a CO₂ storage chamber, and returns the binding agent to the CO₂ capture apparatus.

In a further aspect, the invention provides a method for removing carbon dioxide from atmospheric air that includes the steps of providing a large volume flow of atmospheric air to a CO₂ capture apparatus that includes a CO₂ binding agent, absorbing CO₂ from the large volume flow of atmospheric air to form complexed binding agent, transporting the complexed binding agent to a reprocessing apparatus that releases CO₂ from the complexed binding agent to regenerate the CO₂ binding agent, removing the released CO₂ from the reprocessing apparatus, and returning CO₂ binding agent from the reprocessing apparatus to the CO₂ capture apparatus.

### BRIEF DESCRIPTION OF THE FIGURES

The present invention may be more readily understood by reference to the following drawings wherein:

FIG. 1 provides two views of a system of the invention including a natural draft cooling tower, a CO₂ capture apparatus, and a reprocessing apparatus. FIG. 1A provides a perspective view, FIG. 1B provides a top view, and FIG. 1C provides a bottom view.

FIG. 2 provides a side perspective view of a pseudo-cooling tower and an associated reprocessing apparatus that provides a system for removing atmospheric CO₂.

FIG. 3 provides three views of a wind capture device that includes a CO₂ capture apparatus and is associated with a reprocessing apparatus. FIG. 3A provides a side perspective view of the wind capture device, FIG. 3B provides a front view of the wind capture device, and FIG. 3 C provides a top view of the wind capture device.

FIG. 4 provides a schematic representation of a system for using waste flue gas from a petroleum refinery to heat air in a pseudo cooling tower including a spray column.

FIG. 5 provides a schematic representation of a system for using waste flue gas from a petroleum refinery to heat air in a pseudo cooling tower including a wetted wall.

### DETAILED DESCRIPTION OF THE INVENTION

To illustrate the invention, several embodiments of the invention will now be described in more detail. Reference will be made to the drawings, which are summarized above. Reference numerals will be used to indicate parts and locations in the drawings. The same reference numerals will be used to indicate the same parts or locations throughout the drawing unless otherwise indicated.

### DEFINITIONS

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

The present invention provides systems and methods to harvest the CO₂ present in a given volume of air without having to build a massive and expensive electrical energy infrastructure. In particular, systems and methods have been developed to capture the very dilute CO₂ from atmospheric air at a reasonable cost.

A primary reason why conventional methods for removing CO₂ require so much energy is that while there is very little CO₂ in the air, all of it has to be transported and then treated to remove the CO₂. With such low CO₂ concentration a huge volume of air has to be processed. Accordingly, one way to reduce the amount of energy needed is to separate the adsorbed/bound CO₂ complex from the air or liquid before most of the energy is applied. An addition way to reduce the amount of energy needed is to take advantage of a large existing airflow, or to efficiently generate such an airflow. The present invention therefore provides systems and methods for efficiently processing large volumes of air to remove CO₂, with the bulk of the energy being applied later once the CO₂ has been separated from the air or other gas.

The systems and methods of the invention can be applied to capture of CO₂ from a broad range of gas mixtures. Examples include atmospheric air with very low levels (e.g., 385 ppm) of CO₂ as well as more concentrated sources such as flue gas that typically contain 10 to 15% CO₂. Atmospheric air, as defined herein, is the mixture of gases that would be found in the troposphere, and would typically include about 78% nitrogen, 20% oxygen, 1% argon, and various other gases at smaller quantities, including about 0.038% carbon dioxide. However, atmospheric air is intended to be used in a broad sense to encompass various other air compositions that one can find in terrestrial environments, including those in industrial areas.

The present invention can be used to take advantage of existing airflows in order to process large volumes of air, or the invention may include an apparatus to efficiently generate a large volume airflow. Accordingly, in one aspect, the present invention provides a system for removing carbon dioxide (CO₂) from atmospheric air that includes an apparatus that provides a large volume airflow such as a cooling tower, a pseudo-cooling tower, or a wind capture device. These different apparatus take advantage of different methods for processing a large volume of air, as will be described in greater detail herein. A large volume of air, resulting from a large volume of airflow, can be represented by a range of different volumes of air. For example, a large volume of air can be one thousand tons of air per day, 500 thousand tonnes of air per day, 1 million tons of air per day, or more than one million tons of air per day. As would be understood by one skilled the art, while tons are a measurement of weight rather than volume, the density of air is known and therefore the volume can be readily calculated if the weight is specified.

hi one embodiment of the invention, the large volume of air can be provided through the use of cooling towers. Cooling towers, as defined herein, are industrial-sized equipment used to reduce the temperature of a water stream by extracting heat from water and emitting it into the atmosphere. The one or more cooling towers used in the present invention can be any type of cooling tower, including natural draft cooling towers and/or mechanical cooling towers. A natural draft (i.e., hyperbolic) cooling tower makes use of the difference between ambient air and the hotter air inside the tower as it cools the hot a water stream. As hot air moves upwards through the tower, fresh cool air is drawn into the tower through air inlets at the bottom. Because hot air rises naturally, no fan is required to generate airflow. The towers vary in size and shape, with larger structures being about 200 meters tall and 100 meters in diameter, and can be constructed from a variety of materials, such as wood, fiberglass, steel, or concrete. The hyperbolic cylinder shape is preferred to encourage efficient airflow through the tower.

Mechanical cooling towers include large fans to force or drawn air through circulated water. Water in mechanical cooling towards “fill” surfaces, which increases the contact time between the water and air to maximize heat transfer. Mechanical cooling towers can have a variety of shapes such as lineal, square, or round, and can be provided in groups to give sufficient cooling capacity. Mechanical cooling towers include force draft cooling towers in which the air is blow through the tower by a fan located in an air inlet, and induced draft cooling towers in which air is drawn through the apparatus by a fan.

To provide an example of the volume of air that can be treated by an existing power plant, in a Los Alamos-sized operation, two 1-gigawatt (GW) nuclear power plants and a chemical/gasoline complex requiring 6 cooling towers is available. This complex processes 450 million NmVhr of air and yields about 7,800 tonnes of CO₂/day. Assuming all the cooling towers are of about equal size, a 1-GW plant operation would result in the recovery of about 1.17 M tonnes/yr of CO₂.

The cooling towers can already be in existence, in which case the additional CO₂ removal apparatus are added to or near the cooling tower, or the CO₂ removal apparatus can be included in new cooling towers that are being built. However, because the large volumes of air are being moved for other purposes, little cost is added to move the atmospheric air for CO₂ removal. No new power plants would have to be built to get this airflow. Already existing cooling towers could be retrofitted with the necessary CO₂ absorbing and processing apparatus. Moreover, since these units would be parts of power plants or other energy using facilities (i.e., refineries, petrochemical plants, etc), there will typically also be a great deal of low temperature waste steam available. Thus, in addition to large volumes of airflow, there should also be useful sources of heat (e.g., 130° C. waste heat) that can be provided to facilitate the CO₂ removal process for little additional cost.

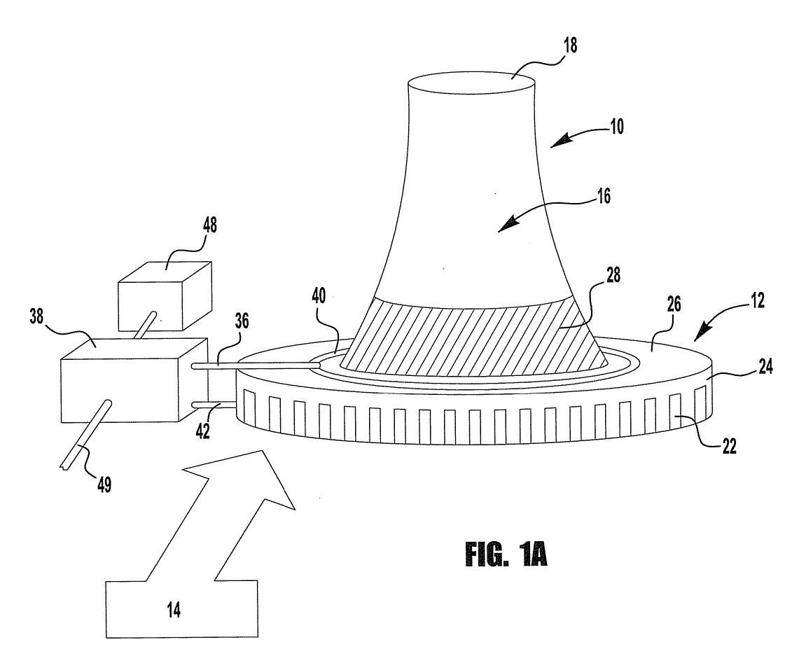

An embodiment of the invention including a natural draft cooling tower is shown in FIG. 1. The system shown includes a natural draft cooling tower 10 and CO₂ capture apparatus 12 positioned to contact atmospheric air 14 moving towards the cooling tower 10, and is positioned between the cooling tower 10 and air inlets 22 that allow entry of atmospheric air 14 into the cooling tower 10. However, in other embodiments, the CO₂ capture apparatus 12 can be positioned to contact atmospheric air moving within the cooling tower 10. The cooling tower 10 is formed from a cooling tower wall 16 that is a generally hollow cylindrical shape forming the cooling tower itself. The tower wall 16 generally includes both a structural frame and a casing around the frame (not shown). The cooling tower 10 has a tower outlet 18 that is an opening at the top end of the cooling tower 10 where processed air leaves the cooling tower 10. The other end of the cooling tower 10 is the tower base 20 which rests on or near the ground at the bottom end of the cooling tower 10.

The CO₂ capture apparatus 12 includes one or more air inlets 22 to allow atmospheric air to enter into the CO2 capture apparatus 12. The air inlets 22 can have a variety of shapes, and can be covered with mesh to prevent debris from entering into the CO₂ capture apparatus 12. In the embodiment shown, the CO₂ capture apparatus 12 includes an entry wall 24 and a capture apparatus roof 26 that runs from the entry wall 24 to the cooling tower wall 16.

The cooling tower 10 also includes a fill 28, which is a warm water distribution apparatus provided within the cooling tower 10 and extending upwards a certain distance from the tower base 20. The fill 28 is a structure within the cooling tower 10 over which warm water from the industrial power source flows, typically as a result of being sprayed from nozzles at the top of the fill 28. The fill 28 has a structure (e.g., successive layers of splash bars) that disperses the water to facilitate heat transfer by increasing the amount of contact between the warm water and the air. As a result of this contact, heat is transferred from the water to the air, causing the air to flow up through the cooling tower, removing heat from the water and generating an airflow. A basin 30, shown in FIG. 1C, is a pool positioned beneath the cooling tower 10 to collect the cooled water that flows off of the fill 28 and other portions of the interior of the cooling tower 10. Warm water flows into the fill 28 through a warm water inlet line 32, which provides hot water from the power plant, and then leaves the cooling tower from the basin 30 through a cooling water outlet line 34.

As noted above, the system of the invention include a CO₂ capture apparatus 12 that is positioned to contact atmospheric air moving towards or within the cooling tower 10. A wide variety of different CO₂ capture apparatus 12 are suitable for use in the present invention, including, for example, fluidized beds, fixed bed reactors, wetted walls, and spray towers. What is most important is that the CO₂ capture apparatus 12 is positioned to take advantage of the airflow generated or intercepted by the cooling tower, pseudo-cooling tower, or wind capture device.

In the embodiment shown in FIG. 1, the CO₂ capture apparatus 12 is positioned around the cooling tower base 20. The CO₂ capture apparatus 12 shown in the figure includes entry wall 24 and a capture apparatus roof 26. CO₂ binding agent is delivered to the CO₂ capture apparatus 12 through a binding agent input line 36 that transfers CO₂ binding agent from the reprocessing apparatus 38. A binding agent distribution line 40 can also be provide, for example along the capture apparatus roof 26, as shown in FIG. 1B, to provide a stream of CO₂ binding agent that will intercept the air flowing through the CO₂ capture apparatus 12.

As noted above, the CO₂ binding agent can be delivered to a plurality of regions within the CO₂ binding apparatus 12 via the binding agent distribution line 40. The CO₂ binding agent mixes with the atmospheric air to form complexed binding agent, which is CO₂ binding agent which has reacted with CO₂, and is then captured in the binding agent basin 44 provided within the CO₂ capture apparatus 12. The CO₂ binding agent can be sprayed down from the binding agent distribution line to form a spray reactor, or it can run down a surface that intersects the airflow in the manner of a wetted wall reactor. After the air has mixed with the CO₂ binding agent, it passes from the CO₂ binding apparatus 12 to the cooling tower 10 through interior air inlets 46 positioned along the cooling tower base 20.

The communication between the reprocessing apparatus 38 and the CO₂ capture apparatus 12 also includes the transfer of complexed binding agent from the CO₂ capture apparatus 12 to the reprocessing apparatus 38 through a complexed binding agent output line 42, which transfers complexed binding agent from the CO₂ capture apparatus 12 to the reprocessing apparatus 38. While single lines are shown for the output line 42 and the input line 36, it should be understood that multiple lines can be used, or the lines may simply represent a point of transfer from one portion of a larger apparatus to another in which CO₂ binding agent binds CO₂ and is then regenerated.

The reprocessing apparatus 38 carries out two main functions; the regeneration of the CO₂ binding agent, and the sequestration of CO₂ released from the binding agent during regeneration of the binding agent. While the method of regenerating the CO₂ binding agent and stimulating CO₂ release varies depending on the nature of the CO₂ binding agent, regeneration of the CO₂ binding agent often involves application of heat to the CO₂ binding agent, such as waste heat having a temperature of 130° C. or more. In some embodiments of the invention, in particular when a cooling tower or pseudo-cooling tower is used to provide a large volume of atmospheric air, the heat is waste heat that is obtained from a proximate industrial power source. The waste heat can be provided to the reprocessing apparatus 38 through a waste heat input line 49. Once the CO₂ has been released from the binding agent, it is directed to a CO₂ storage site 48. The storage site 48 may provide temporary storage until the CO₂ is used for another purpose, such as oil recovery or the stimulation of plant growth, or the storage site 48 may involve long-term storage of the CO₂, such as sequestration to an underground site or liquefaction for compact storage under pressure.

As noted herein, two examples of reprocessing apparatus 38 are fluidized and fixed-bed reactors. Two representative examples of such process configurations are described herein, although it would be clear to one skilled in the art that many more are available. The first configuration comprises two or more fixed bed reactors. In the first reactor air enters at a temperature of about 25° C. and comes in contact with the CO₂ binding agent, such as an immobilized amine. The process preferably continues until that adsorptive capacity of the CO₂ binding agent has been fully utilized, although in some embodiments only a portion of the absorptive capacity of the CO₂ binding agent is utilized. At that point the CO₂ laden air is diverted to the second reactor while the first reactor is evacuated of air and its temperature is increased to about 130° C., at which temperature the CO₂ is desorbed and ready to be used, sequestered, etc., while the reactor is ready to return to adsorb further CO₂. Note that in this embodiment of the invention, the CO₂ capture apparatus 12 and the reprocessing apparatus 38 are coextensive.

The second example of a CO₂ capture apparatus is a fluidized bed configuration. In a fluidized bed configuration, the CO₂ binding agent (e.g., a solid or immobilized binding agent) consists of particles small enough to be suspend in a stream of 25° C. air that is constantly passing through the chamber. During passage through the chamber the particles adsorb the CO₂ which they contact. The reactor is constructed so that essentially all of the particles remain in the chamber, except for a portion which pass through an air lock that allows them, but not the air, to enter another chamber held at a higher temperature (e.g., about 130° C.) where CO₂ is released and sent to a storage area, while the regenerated CO₂ binding agent particles are return to the fluidized reactor. In this process, only the CO₂— saturated CO₂ binding agent is exposed to the higher temperature. It will therefore only require a small fraction of the energy that would otherwise be required.

While passing atmospheric air through a fluidized and fixed-bed reactor filled with saturated CO₂ binding agent is one method for facilitating capture of CO₂, it is not the only way that this system would be operated. Indeed, in some circumstances, passing atmospheric air through this type of a bath might slow down the reaction rate because of its limited surface area. Moreover it might also significantly increase the pressure drop which might in turn require more energy to maintain the required throughput of air through the reactor. Therefore, in some embodiments of this system it might be preferable to use equipment such as spray towers and/or spray chambers in order to rapidly bring and mix air into intimate with a solution including the CO₂ binding agent.

Spray towers and spray chambers, also known as wet scrubbers, are well known in the art. Typically they are empty cylindrical vessels made of steel or plastic with nozzles that spray liquid into the vessels. The inlet gas stream usually enters the bottom of the tower and moves upward, while liquid is sprayed downward from one or more levels. The flow of inlet gas and liquid in the opposite direction is called countercurrent flow. However, a variety of other configurations of spray towers or spray chambers may also be used, including those using co-current or crosscurrent configurations. For a description of additional spray towers and spray chambers, and their use for gas absorption, see “Air Pollution Control: A design approach” 2nd Ed., by C. Cooper and F. Alley, Chapter 7, pgs. 217-246, and Chapter 13, pgs. 411-447, Waveland Press, Inc., 1994, the disclosure of which is incorporated herein by reference. Spray towers or spray chambers could be used to remove CO₂ using any of the reaction systems described herein, such as K₂CO₃, Na₂CO₃ and others.

Spray towers are constructed in various sizes; small ones to handle small gas flows of 0.05 mVs (106 ftVmin) or less, and large ones to handle large exhaust flows of 50 mVs (106,000 rnVmin) or more. Because of the low gas velocity required, units handling large gas flow rates tend to be large in size. The operating characteristics of spray towers are presented in Table 1.

TABLE 1
Operating characteristics of spray towers

| | | | Liquid- | | |
|---|---|---|---|---|---|
| | | | inlet | | |
| | Pressure | Liquid-to-gas | pressure | Removal | |
| Pollutant | drop (Δp) | ratio (L/G) | (p_{L}) | efficiency | Applications |
| Gases | 1.3-7.6 cm | 0.07-2.70 l/m³ | 70-2,800 kPa | 50-90⁺% (high | Mining |
| | of water | (0.5-20 gal/ | | efficiency only | industries |
| | | 1,000 ft³ | | when the gas is | Chemical |
| | | | | very soluble) | process |
| Particles | 0.5-3.0 in | 5 gal/1,000 ft³ is | 10-400 psig | 2-8 μm diameter | industry |
| | of water | normal; >10 | | | Boilers and |
| | | when using | | | incinerators |
| | | pressure sprays | | | Iron and steel |
| | | | | | industry |

Increasing reaction rates by increasing capture surface area (e.g. by creating small liquid droplets, etc) is one way, but not the only way, to increase reaction rates for the capture of CO₂ by reversible salt CO₂ binding agents. Several other approaches have been investigated and at least two seem to hold promise.

The system of the present invention can use a variety of different types of CO₂ binding agents. For example, the CO₂ binding agent can be a compound capable of absorbing CO₂ at a first temperature and then releasing the CO₂ at a second, higher temperature. In one embodiment, the CO₂ binding agent is an immobilized amine capable of absorbing CO₂ at a first temperature and releasing the CO₂ at a second higher temperature, while in other embodiments the CO₂ binding agent is a salt capable of absorbing CO₂ at a first temperature and releasing the CO₂ at a second higher temperature.

In one embodiment of the invention, the CO₂ binding agent is an amine such as an immobilized amine. The use of amines as CO₂ binding agents has been described by Steven S. C. Chuang, et al. in Ind. Eng Chem. Res 2005, 44, p. 3702-3708, the disclosure of which is incorporated herein by reference. Chuang describes how an amine with high CO₂ adsorption capacity can be grafted into SBA-15 creating a solid reversible product that will adsorb CO₂ at 25-30° C. and desorbs it at about 120° C. While this material was developed to remove CO₂ from flue gas (where CO₂ is usually present in a concentration from about 10-15%) it would also work to capture CO₂ that is present in far lower concentrations, such as those found in atmospheric air. Various alkyl and aryl amines are suitable, with primary amines being preferred.

hi another embodiment of the invention, CO₂ removal from air is based on the reaction of CO₂ with a concentrated K₂CO₃ (potassium carbonate) solution. The reaction is summarized by following equation:

K₂CO₃+CO₂+H₂O→2KHCO₃ [Eq 2]

in this embodiment of the system, CO₂ removal involves a number of steps. First, atmospheric air is run through a CO₂ capture apparatus contains a concentrated K₂CO₃ solution The solubility OfK₂CO₃ in water at 20° C. is 112 g/100 mL. Since the solubility of KHCO₃ in water at 20° C. is 22.4 g/100 mL it will typically precipitate from solution upon capture of CO₂. One advantage to precipitating the bicarbonate is that much less water is present, and therefore less energy is required to move the complexed binding agent and eventually disassociate CO₂ from the binding agent to regenerate the CO₂ binding agent. Typically, the complexed binding agent is precipitated when a spray reactor CO₂ removal apparatus is used, while it is preferable that the complexed binding agent remain in solution in a wetted wall CO₂ removal apparatus.

While there are advantages to precipitating the complexed binding agent (e.g., KHCO₃), depending on the temperature and the amount of exposure to CO₂, the potassium bicarbonate may not precipitate in some embodiments of the invention, but rather simply increases in concentration in solution in response to the exposure to CO₂. The KHCO₃ (either precipitated or in solution) is then transferred to the reprocessing apparatus where the it is heated to between about 100 and 200° C., causing it to decompose, thereby regenerating CO₂ gas and K₂CO₃. The temperature determines the rate of decomposition. Decomposition occurs as indicated by the following equation:

2KHCO₃→K₂CO₃+CO₂T+H₂O [Eq 3]

This process has a number of advantageous features. Since very little water is carried along with the precipitated K₂CO₃ the energy to move, capture and recover the CO₂ is low. Also, because the reactions are carried out at relatively low temperature and ambient pressure the cost of the equipment is low without any need for expensive materials of construction. Moreover, since carbonate and bicarbonate are relatively innocuous, the equipment and protective gear required can be expected to be relatively simple and inexpensive. In addition, the price for bulk potassium carbonate is low, i.e., well below $1/lb, which further decreases the cost of operating the system, especially since it can be recycled and reused numerous times.

One advantage OfK₂CO₃ as a CO₂ binding agent is that it is not degraded or oxidized when exposed to the contemplated processing conditions. It can therefore be recycled numerous times, further lowering the operating cost of the system. Also while some potassium carbonate may cling to the precipitated bicarbonate, it should not significantly affect the reaction. While amines can be regenerated and reused, they tend to become degraded over time and need to be replaced.

As noted in Equation 3, it takes a mol of potassium carbonate to capture and release one mol of CO₂. This is equivalent to about a 32% wt/wt of CO₂ capture loading which compares favorably with other CO₂ binding agents.

The economics of this system also depends on the rate of reaction OfK₂CO₃ with CO₂ which is satisfactory but amenable be further improvement. Methods that can be used to further improve the reaction rate are further discussed herein.

K₂CO₃ will react with strong acid gasses (e.g., H₂S, SO₂. HCl) to form stable irreversible salts that will not decomposed under normal conditions and cannot be separated and recover from the remaining K₂CO₃. When this occurs the quality of the solvent is degraded, the efficiency of the process is reduced and the cost of the operation increased. This is not a significant problem when the carbonate is used to remove CO₂ from atmospheric air since these gasses are rarely found in significant quantities in the air. However, it can be a problem should potassium carbonate be used to remove CO₂ from inadequately treated flue gas. While this could create a problem, one or more solutions are available, as will be later discussed herein in the context of CO₂ removal from flue gas.

While the foregoing has focused on the use of potassium carbonate as a CO₂ binding agent, other materials can be used in the manner describe above to remove and recover a relatively pure stream of CO₂. For example it is possible to use Na₂CO₃ as a CO₂ binding agent in a system substantially equivalent to that described above, since the solubility of sodium carbonate and bicarbonate are 30 g/100 mL and 7.8 g/100 mL respectively, thus allow it to be used effectively as an adsorbent in such a system. Similarly Cs₂CO₃ (cesium carbonate) could also scavenge CO₂. Here again these compounds have appropriate solubility characteristic (i.e., 326 g/100 mL for Cs₂CO₃ and 209 g/100 mL for CsHCO₃) which would allow them to operation in an analogous fashion. Indeed any solvent which can reversible combine with CO₂ and where there is a reasonable difference in the solubility between the solvent itself and the solvent once it combines with CO₂ would operate in a similar manner and produce results equivalent to those described above.

In addition to a CO₂ binding agent, the system for removing CO₂ from atmospheric air or point sources such as flue gas can also include a catalyst (e.g., an enzyme) that improves the kinetics of CO₂ absorption. Examples of catalysts that may be used in the system include piperazine, carbonic anhydrase enzyme, or a synthetic carbonic anhydrase enzyme analog.

It is generally recognized that amines react more rapidly with CO₂ than do alkaline salts such as K₂CO₃. Moreover, it has been found that adding catalytic quantities of amine appears to increase the speed of reaction of K₂CO₃ and other similar compounds. Of these amines it has been reported that piperazine appears to be the strongest promoter able to accelerate the absorption CO₂ by as much as a factor of three.

Another approach focuses on the use carbonic anhydrase one of the most powerful substances known to catalyze the transformation of CO₂ into bicarbonate ions. When considering the carbon capture reaction more carefully it should be noted that two sequential reactions are really involved: (1) CO₂+CO₃^{r}H₂O→2HCO₃ followed by 2 HCO₃″″+2K⁺→2 KHCO₃. The first of these reactions is the slower of the two, so if it can be speeded up, the overall reaction would be accelerated. It has been reported that this have been able to increase the overall rate of reaction by a factor of 5 to 6.

Even though there is a large inventory of cooling towers and more will be built as the need for energy grows it is possible that even more CO₂ capture capacity from the atmosphere might be required. Accordingly, the inventors have developed an additional apparatus that can be used to efficiently provide a large volume of air for removal of carbon dioxide from atmospheric air. This aspect of the system includes the use of a pseudo-cooling tower. A pseudo-cooling tower, as defined herein, is a cooling tower-like structure that provide a large volume airflow as a result of heating air within a column to cause it to rise and create an air current in a manner similar to that in a conventional natural draft cooling tower, but without using hot water from an industrial power source as the source of heat. Instead, the pseudo-cooling tower provides heat to the air within the tower from flare gas, which is provided as hot air that has already been burned, or which is burned within the tower, to generate hot air.

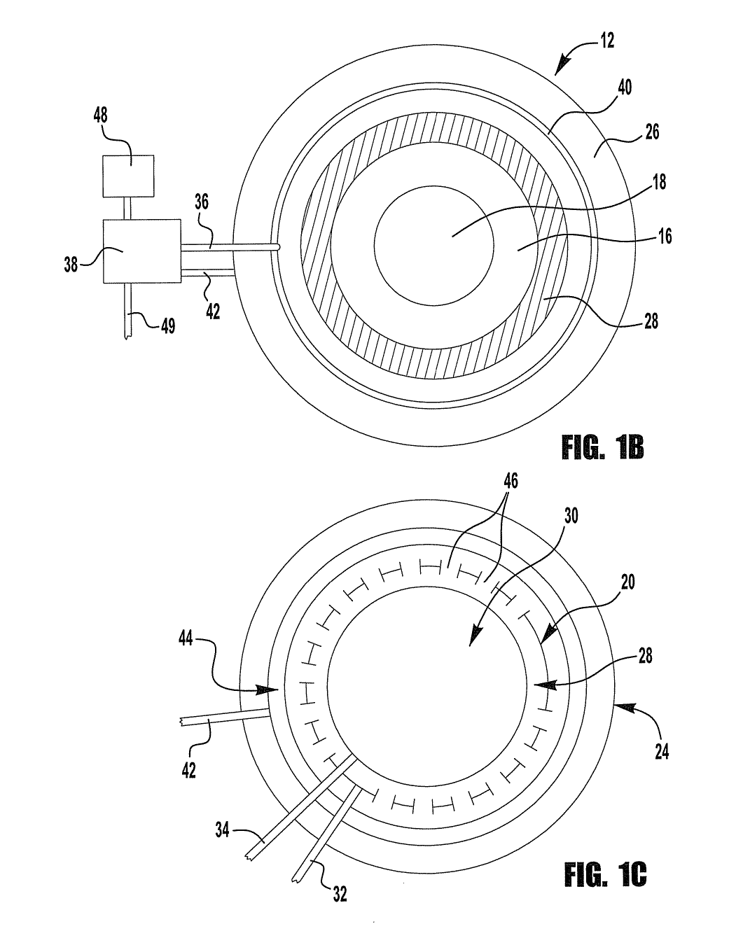

An example of a pseudo-cooling tower is shown in FIG. 2, which provides a side perspective view of a pseudo-cooling tower 50. The pseudo-cooling tower 50 makes use of many of the same components as a standard cooling tower. For example, the pseudo-cooling tower includes a tower wall 16 that provides the basic structure of the pseudo-cooling tower, a tower outlet 18 at the top of the tower, and a tower base 20 at the bottom of the tower. The pseudo-cooling tower also includes an air inlet 22 at the at the tower base 20. In the same manner as with the interior air inlets 46 of a cooling tower 10, the air inlet 22 can be created by supporting the tower base 20 on struts 52 to provide a gap through which atmospheric air can be drawn into the pseudo-cooling tower 50. While pseudo-cooling towers 50 are similar to cooling towers 10, in some embodiments they can be shorter, simpler and made of less expensive materials.

As already noted, the main way in which pseudo-cooling towers 50 differ from cooling towers 10 is that the heat needed to drive the tower is provided by waste heat such as that provided by flare gas rather than being provided by hot water from an industrial power source. Flare gas includes gaseous hydrocarbon fuels such as methane. A CO₂ removal system using a pseudo-cooling tower will therefore also include one or more flare gas input lines 54 that provide flare gas from the industrial power source to the pseudo-cooling tower 50. The flare gas input line 54 provides flare gas to the flare gas burners/outlets 56 that are supported on the tower wall 16 by an outlet support apparatus 58. The flare gas burners/outlets 56 can either release hot gas from burnt flare gas, in the case of outlets, or an actual flame of burning flare gas, in the case of burners, hi either case, the flame or hot gas is directed upwards into the interior of the pseudo-cooling tower. While a single flare gas burner/outlet 56 can be provided, typically a plurality of flare gas burner/outlets are provided within the pseudo-cooling tower 50. The outlet support apparatus 58 is one or more struts that support the one or more flare gas burner/outlets 56 within the pseudo-cooling tower, and are attached to the interior of the pseudo-cooling tower 50. While the flare gas burners/outlets 56 can be positioned at various heights within the pseudo-cooling tower 50, in a preferred embodiment the flare gas burners/outlets 56 are positioned within the upper half of the tower.

Just as in the conventional natural draft cooling towers the air at the top of the pseudo-cooling tower will be hotter and the density lowers at the top than the air at the bottom. This difference in density would be the driving force that will draw atmospheric air into and through the pseudo-cooling tower, hi a natural draft tower, the heat is supplied by the hot process water that is sent to the tower for cooling. Similarly the heat supplied to the air within the pseudo-cooling tower would drive the process.

The pseudo-cooling tower 50 can be used with a variety of CO₂ capture apparatus 12. Examples of suitable CO₂ capture apparatus 12 include fluidized beds, fixed bed reactors, wetted walls, and spray towers. The embodiment of a pseudo-cooling tower 50 provided in FIG. 2 includes a wetted wall CO₂ capture apparatus 12. However, a variety of CO₂ capture apparatus can be used.

The wetted wall CO₂ capture apparatus 12 provided in the embodiment shown in FIG. 2 includes a plurality of release apparatus 60 supported within the tower wall 16 by the first-release support 61. The release apparatus 60 can be nozzles such as spray nozzles that direct a solution including the CO₂ binding agent down along the interior of the tower walls 16. The first release support 61 is attached within the tower walls 16 and holds the release apparatus 60 in position. In this configuration the CO₂ binding agent solution would flow down within the pseudo-cooling tower in a direction counter to the air flow on wetted wall slats 64 positioned within the pseudo-cooling tower 50. Note that while a single release apparatus 60 and support 61 can be used, additional release apparatus 60 and a second release support 62 can also be included within the pseudo-cooling tower 50. For example, as shown in FIG. 2, in one embodiment, the pseudo-cooling tower 50 includes release apparatus 60 and a first release support 61 above the flare gas burner/outlets 56, and additional release apparatus 60 and a second release support 62 below the flare gas burner/outlets 56. When more than one set of release apparatus 60 and supports 61 and 62 are used, a binding agent transfer line 65 may be included to provide binding agent to the additional release apparatus 60. If a release apparatus 60 is provided above the flare gas burners/outlets 56, it may be preferable to provide cover plates 66 over the flare gas burner/outlets 56 to prevent them from being doused or clogged with the CO₂ absorbing agent solution that flows released from the release apparatus 60.

The solution including the CO₂ binding agent that is released within the pseudo-cooling tower 50 is eventually collected in the binding agent basin 44 provided at the bottom of the pseudo-cooling tower 50. The CO₂ binding agent, which is primarily complexed binding agent at this point, is then withdrawn from the basin and transferred to the reprocessing apparatus 38 via one or more complexed binding agent outlets 42. As described earlier herein, the reprocessing apparatus releases and stores the CO₂ and returns regenerated CO₂ binding agent to the pseudo-cooling tower 50 through a binding agent input line 36. Note that water or other solvent may need to be added to the regenerated CO₂ binding agent before it is returned to the cooling tower or pseudo-cooling tower.

Waste heat available from industrial plants such as steel mills, cement plants, oil refineries, petrochemical plants, etc. could be economical sources of energy which could be used to heat the air at the top of the pseudo-cooling tower. These sources of low grade heat which are produced in significant quantities throughout the world would allow the capture large quantities of CO₂ from the atmosphere. Moreover since these sources of waste are available at relatively low cost this method of capturing CO₂ should be only slightly more expensive than that will use cooling towers. To provide some additional information related with such a system when operated at a hypothetical 150,000 bb I/day oil refinery, a preliminary estimate of the cost of capturing CO₂ from the air as well as its mass & energy balance is provided in Examples I and II, herein.

The energy for heating the air at the top of the pseudo-cooling tower could also be provided by burning natural gas, biomass or coal. CO₂ is preferably removed from both the air and the combustion gases resulting from burning natural gas, biomass, or coal to produce the heat. The advantage of this method is that it would make the operation completely independent of any other industrial facilities {i.e. power generating plants or other sources of waste heat). However, the cost of capturing CO₂ in this fashion would be higher due to higher infrastructure and operating costs.

Another aspect of the invention allows the removal of CO₂ from atmospheric air where there is a significant amount of wind energy available, and in particular where an industrial power source is unavailable. In such situations, wind energy can be used to provide a large volume of atmospheric air to allow the removal of the CO₂ from the air in a relatively efficient and inexpensive manner.

While wind is free, it does have some shortcomings. These include that wind does not typically blow at a constant rate, that available wind power is not always strong enough to move the large amount of air needed in a reasonable period of time to make the capture of atmospheric CO₂ practical, and that operating expenses will be higher compared to facilities that can use cooling towers or pseudo-cooling towers, particularly those that have waste heat available which can be used to regenerate the CO₂ binding agent and provide motive power. However, should a wind-driven CO₂ removal system be co-located with a wind farm, it might be able to obtain power to run the reprocessing apparatus from the wind farm.

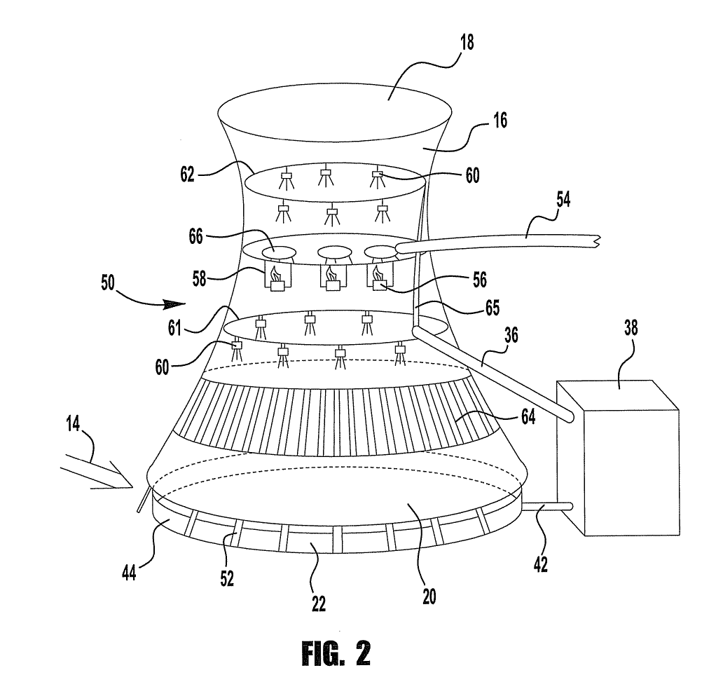

A wind capture device capable of directing a large volume of atmospheric air into contact with a CO₂ capture apparatus can have a wide variety of configurations, depending in part on the nature of the CO₂ capture apparatus. However, regardless of the particular shape chosen, the wind capture device must be open on the side intended to face the wind, and also have an opening, preferably on the opposite side, to allow wind entering the wind capture device to rapidly flow through the device while providing contact with the CO₂ absorbing apparatus. One embodiment of a wind capture device that can be used for the system for removing CO₂ of the present invention is shown in FIG. 3. The wind capture device 70 is a rectangular box that includes a rectangular binding agent reservoir 72 positioned in parallel and overlapping a rectangular receiving reservoir 74. The binding agent reservoir 72 is formed by an upper reservoir enclosure 76 that includes a front, a back, and two sides that run along the perimeter of the reservoir. The receiving reservoir 74 is formed by a lower reservoir enclosure 78 that includes front, a back, and two sides that run along the perimeter of the reservoir. The two sides of the upper reservoir enclosure 78 are connected to the two sides of the lower reservoir enclosure 78 by sidewalls 80.

Multiple flowposts 82 are included within the wind capture device 70. One end of each of the flowposts 82 is connected to the binding agent reservoir 72, while the other end is connected to the receiving reservoir 74. The flowposts 82 are designed to allow the CO₂ binding agent solution 84, i.e., a solution including a CO₂ binding agent, to flow down from the binding agent reservoir 72 along the surface of the flowpost 82 into the receiving reservoir 74 at a moderate rate that allows the atmospheric air flowing into the wind capture device 70 from the opening at the front of the device to have significant contact with the CO₂ binding agent solution 84 before flowing out from the wind capture device 70 through an opening at the back of the device.

The upper end of the flowposts 82, or the point at which they connect to the binding agent reservoir 72, is designed to allow the flow of the CO₂ binding agent solution 84 out from the binding agent reservoir 72 and along the surface of the flowposts 82, as shown in FIG. 3B. This can be achieved in a variety of ways. For example, in one embodiment, the flowposts 82 can include a cavity positioned in the top of the flowposts 82 where they connect to the binding agent reservoir 72 and a plurality of openings along the cavity that allow the CO₂ binding agent solution 84 to flow into and then out from a top region of the flowposts 82 (not shown). In another embodiment, a flow opening 86 is provided in the binding agent reservoir 72 adjacent to the point where the flowposts 82 are connected to the binding agent reservoir 72 by one or more attachments 88 that secure the upper region of the flowposts 82 to the binding agent reservoir 72, as shown most clearly in FIG. 3C. The flow opening 86 should have a size that provides a slow flow of CO₂ binding agent solution 84 from the binding agent reservoir 72 and along the flowposts 82.

The wind capture device includes a CO₂ capture apparatus, which consists of the reservoirs, flowposts, and CO₂ binding agent solution, that are integrated into the wind capture device itself. This CO₂ capture apparatus is in communication with a reprocessing apparatus 38 that releases CO2 from the complexed binding agent, stores the CO₂, and regenerates the CO₂ binding agent. Accordingly, the reprocessing apparatus 38 is connected to the wind capture device 70 through a complexed binding agent outlet 42, which transfers complexed binding agent from the receiving reservoir 74 to the reprocessing apparatus 38, and a CO₂ binding agent inlet 36, which transfers regenerated CO₂ binding agent back to the binding agent reservoir 72. Note that the CO₂ binding agent may need to be resuspended in solution after being regenerated, depending on how the reprocessing is carried out.

As illustrated in Example 4, it may be preferable to provide a system that includes a plurality of wind capture devices 70 in order to reduce the size of the individual devices. The wind capture devices 70 are shown in FIG. 3 as open rectangular boxes, but other shapes can be used. Preferably, these open boxes would have to be reasonable sturdy and deep enough to be self-supporting and firmly attached to the CO₂ capture devices. To be stable it is estimated that these units (“boxes”) would have to be about 15 ft deep. Thus, based on these assumptions, and that 25 of these 300×60×15 ft boxes would be required to replicate the CO₂ capture capability of a 1 GW power plant, about 270,000 square feet of metal or plastic material would be required, to build these boxes. Moreover, these structures should preferably be elevated {e.g., by about 30 to 45 ft) above ground level, which would require additional material and fabrication costs.

A system for removing atmospheric CO₂ using wind capture devices runs primarily on wind power and gravity, as described. However, some energy still needs to be provided to carry out processes such as regenerating the CO₂ binding agent. Accordingly, in some embodiments of the system it may be preferable to locate the wind capture devices proximate to a wind turbine that can provide the energy needed to operate one or more components of the system, such s the reprocessing apparatus.

The present invention also includes a system for removing carbon dioxide from flue gas. Flue gas represents a more concentrated source of CO₂ and therefore represents an excellent opportunity to remove CO₂ from a gas stream before it enters the atmosphere. A system for removing CO₂ from flue gas will include a CO₂ capture apparatus positioned to contact flue gas moving from or within a smokestack and a reprocessing apparatus in communication with the CO₂ capture apparatus. The CO₂ capture apparatus and reprocessing apparatus can be essentially the same as any of those described for the capture of CO₂ from atmospheric air using cooling towers or pseudo-cooling towers. The CO₂ capture apparatus includes a CO₂ binding agent that binds to CO₂ in atmospheric air, and the reprocessing apparatus releases CO₂ from the binding agent, directs the released CO₂ to a CO₂ storage chamber, and returns the binding agent to the CO₂ capture apparatus, as described.

As previously noted the technology described herein can not only remove CO₂ from the atmosphere but for flue gas as well. Indeed since as a rule flue gas contains 10 to 15% CO₂ while there is less than 400 ppm in the air, the extraction should be much easier. However, the removal of CO₂ from flue gas can be complicated by the presence of contaminants that vary from flue gas to flue gas. Indeed no two flue gases are exactly the same because of the energy source (e.g., varying types of coal) that is used and the way that it is processed can be substantially different. Thus for example consider the composition of a flue gas resulting from the burning of coal, shown in Table 2:

TABLE 2

| | N₂ | O₂ | CO₂ | SO₂ | SO₃ | NOx | HCl | Moisture |
|---|---|---|---|---|---|---|---|---|
| Vol % | 73.8 | 4.8 | 12.3 | 0.24 | 0.0024 | 0.06 | 0.01 | 8.8 |

If the coal is properly cleaned (i.e. SOx, NOx & HCl) and dried. CO₂ can be readily removed from the flue gas using the processes described herein. But since K₂CO₃ (like all other solvents) irreversibly combine with these impurities to form compounds that cannot be easily separated from the solvent it may be necessary to pretreat the flue gas before it enters the process. Also if there is a significant amount of water vapor in the gas it might be most cost effective to first remove the moisture from the flue gas. One relatively inexpensive way to do this would be to cool down the flue gas (which usually arrives at 120 to 140° F.) to condense the water. Since the acid gasses (i.e., SOx, NOx & HCl) are quite soluble in water that would also remove them from the flue gas thus not only reducing the amount of material that would have to be processed but also insuring that these deleterious materials are removed as well.

In one embodiment of the system for removing CO₂ from flue gas, the CO₂ binding agent is potassium carbonate. In another embodiment, the CO₂ capture apparatus comprises a spray tower, hi a further embodiment, the CO₂ capture apparatus includes a wetted wall. If a wetted wall is used, it is preferable that the binding agent is potassium carbonate, and the binding agent is provided in an aqueous solution and does not precipitate during the capture or release of CO₂. An example of such a system includes one in which the potassium carbonate forms potassium bicarbonate upon binding of CO₂, the concentration of the potassium bicarbonate in the aqueous solution is from about 25% to about 35% after absorption of CO₂ at a first temperature, and the concentration of potassium carbonate is from about 15% to about 25% after release of CO₂ at a second higher temperature. However, in alternate embodiments of the system for removing CO₂ from flue gas, the CO₂ binding agent can precipitate from an aqueous solution after absorbing CO₂.

In another aspect, the present invention provides a method for removing carbon dioxide (CO₂) from atmospheric air that includes the steps of providing a large volume flow of atmospheric air to a CO₂ capture apparatus that includes a CO₂ binding agent, absorbing CO₂ from the large volume flow of atmospheric air to form complexed binding agent, and transporting the complexed binding agent to a reprocessing apparatus that releases CO₂ from the complexed binding agent to regenerate the CO₂ binding agent.

Regeneration of the CO₂ binding agent refers to reforming the original chemical that was used as the binding agent, such as converting potassium bicarbonate back to potassium carbonate. The released CO₂ is then removed from the reprocessing apparatus, and the regenerated CO₂ binding agent is transferred from the reprocessing apparatus to the CO₂ capture apparatus. In some embodiments of the invention, the CO₂ is released from the complexed binding agent by heating the complexed binding agent, hi further embodiments, the heat used to heat the complexed binding agent is provided by waste heat from a proximal industrial power source.

The method for removing carbon dioxide from atmospheric air can also further include the step of storing the CO₂ released from the reprocessing apparatus. As noted previously herein, the CO₂ may be stored temporarily until it is used for another purpose, such as underground petroleum recovery or the stimulation of plant growth (e.g., algal growth), or the CO₂ may be placed in long-term storage, by, for example sequestering the CO₂ in an underground site or liquefying the CO₂ to facilitate compact storage under pressure.

The method for removing CO₂ from atmospheric air can be used to remove C02 from a large volume of atmospheric air. As described earlier herein, a large volume of air can represent a variety of different volumes of air. For example, a large volume of air can be one thousand tons of air per day, 500 thousand tonnes of air per day, 1 million tons of air per day, or more than one million tons of air per, day, or any of the amounts of air processed in the examples described herein. Depending on the efficiency of the removal of CO₂, and the amount of atmospheric air processed, a range of different amounts of CO₂ can be removed from atmospheric air. For example, embodiments of the invention can remove about 250 tons of CO₂ per day, about 500 tons of CO₂ per day, or about 1000 or more tons of CO₂ per day.

The 2006 US inventory of electrical plants (see Table 3 below) provides the data can be used to determine the amount of CO₂ which could be removed using an embodiment of the method described herein.

TABLE 3

| | Number | Generator Nameplate | Avg Generator |
|---|---|---|---|
| Energy Source | of Generators | Capacity (in MW) | Size (in MW) |
| Nuclear | 104 | 105,585 | 1006 |
| Coal | 1493 | 335,830 | 225 |
| Natural Gas | 5470 | 442,945 | 81 |

The plants that utilize these three “energy sources” account for about 82% of the total US electrical generating capacity. Of these, nuclear and coal-based energy sources usually supply the base load and therefore operate at nearly full capacity year around. It is also estimated that at present plants with about 50% of the available electrical generating capacity use cooling towers. Based on these estimates it appears that existing US electrical utility plants that use cooling towers would be able to capture about 491.4 M tonnes/yr of atmospheric CO₂ if provided with the CO2 removal system of the present invention.

The systems and methods of the present invention are able to capture CO₂ at a relatively low cost from gas streams regardless of its concentration, even at concentrations as low as that in the atmosphere. The energy requirements are modest since CO₂ would be removed at ambient temperature and pressure from gas streams which already would be moving for some other purpose (e.g., cooling tower air, flue gas exhaust). Therefore, the only extra energy needed would be that required to compensate for the added pressure drop resulting from the air having to pass through the aqueous solution; and the energy to move the precipitated solid from the first to the second reactor, though much of it could be supplied by gravity. In addition, the temperature of the precipitated solid can be raised to a temperature in the range of about 100 to about 200° C. to recover the CO₂. Considering that these systems would typically be associated with high energy consuming or production plants (e.g., power plants) low grade steam should be available, inexpensive and plentiful. Moreover, heat exchangers could even recover much of that energy. Energy would also have to be expended to return the recovered K₂CO₃ to the first reactor; but this energy requirement should be minimal. Finally, CO₂ transport would require little energy unless it has to be pressurized or liquefied for storage or sequestration, or for use by some other end-use purpose.

In order to have a better understanding of this system four examples have been included to more clearly describe particular embodiments of the invention and their associated cost and operational advantages. However, there are a wide variety of other embodiments within the scope of the present invention, which should not be limited to the particular examples provided herein.

### EXAMPLES

### Example 1

### Plant Configurations Using Waste Heat from Oil Refinery Flare Gas & “Pseudo Cooling Towers” to Capture Coτusing an Absorber/Spray Column

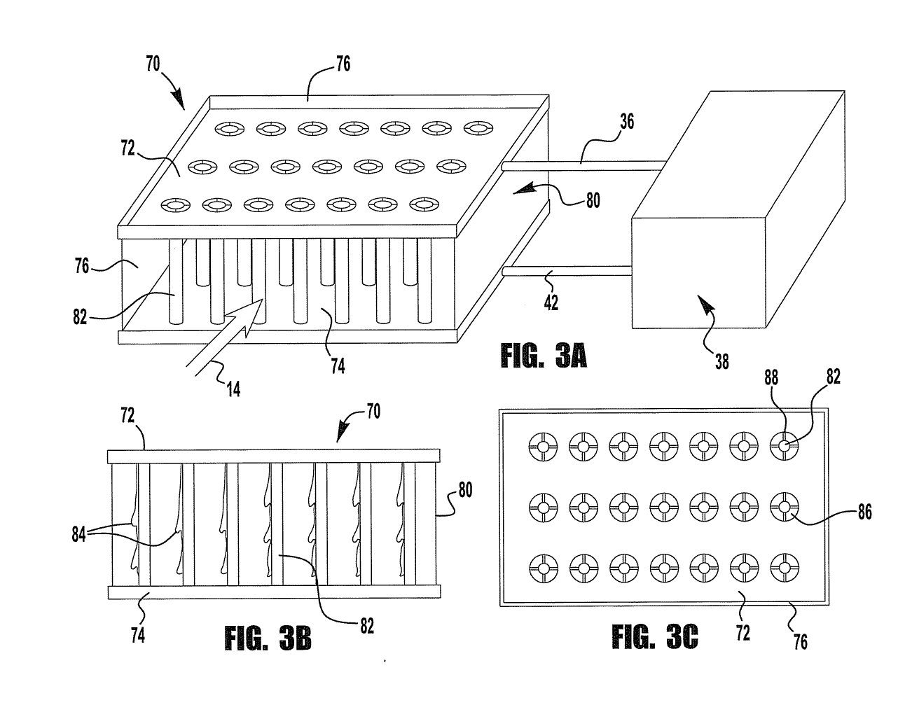

This example describes the use of waste flue gas from an industrial power source (e.g., a refinery) to heat air in a pseudo cooling tower constructed to simulate the flow of atmospheric air in a power plant cooling tower. A schematic representation of this embodiment of the invention is shown in FIG. 4. The flare gas is burned with a separate source of air by burners that are inserted in the middle section of the pseudo-cooling tower. The combustion gases mix and heat the air towards the top section of the pseudo-cooling tower 50 thus reducing the density of the gases at the top pseudo-cooling tower and causing the air to flow up, drawing atmospheric air 14 into the pseudo-cooling tower 50. At the same time a concentrated K₂CO₃ solution is sprayed down from the top the pseudo-cooling tower, allowing it to absorb the CO₂ from the rising air and combustion gases in the CO₂ capture apparatus 12 {e.g., a spray column).

Assuming a 20° F. (11.1° C.) increase in temperature from the bottom to the top of the pseudo-cooling tower 50, the heat of combustion of the flare gases from a 150,000 bbl/day refinery would provide enough energy to move about 10.4 million ton/day of air and other gases through the pseudo-cooling tower. Thus the CO₂ concentration at the top of the pseudo-cooling tower would amount to about 1277 parts per million by weight (ppmw).

In pseudo-cooling tower 50, the K₂CO₃ in solution absorbs the CO₂ and the KHCO₃ precipitates out of solution and some of the water is evaporated. The KHCO₃ precipitates and the solution is then pumped to a reprocessing apparatus 38 (e.g., a desorber/dryer such as a rotary kiln) using a pump 90. In the reprocessing apparatus 38 all of the KHCO₃ precipitates out of solution and the remaining water is evaporated. The wet slurry is then indirectly heated to 160° C. with waste heat 92 (e.g., 150 psig steam obtained from the refinery), decomposing the KHCO₃ and evolving gaseous CO₂ and water.

The CO₂ and water generated by the decomposition of the bicarbonate is sent to the condenser 94 where the water is removed and recycled to the dissolver 96. The hot K₂CO₃ is redissolved in water coming from the dissolver 96 and the resulting solution K₂CO₃ is returned as a concentrated solution sprayed in at the top of the pseudo-cooling tower 50 to capture the CO₂. The CO₂ captured from a 150,000 bbl/day refinery by this process amounts to about 11,800 tonnes per day (T/D) which consists of about 5,440 T/D (46%) from the air and 6,375 T/D (54%) from flue gas combustion. The recovered CO₂ is then compressed, liquefied and pumped to the closest suitable underground geological sequestration sites.

Operating Conditions for the above-described example are as follows:

Flue gas from a 150,000 refinery is used to drive the atmospheric air through the pseudo-cooling tower. It is assumed that a 20° F. (11.1° C.) temperature differential in the pseudo-cooling tower is sufficient to drive the reaction. The induced air flow is about 10.4×10⁶ T/D in the required number of pseudo-cooling towers. The flue gas heating value is 1583 BTU/ft³ with CO₂ concentration of about 15.1%. The calculations further assume a 90% capture of CO₂ from atmospheric air and combustion gas (combined CO₂ concentration of 1277 ppmw). hi addition, the K₂CO₃ loss is assumed to be about 0.1%.

TABLE 4
Material Balance (in Tons/Day)

| Equipment | Material | Inputs | Outputs |
|---|---|---|---|
| Absorber/Spray | Ambient Air (w 385 ppm CO₂) | 10.4 × 10⁶ | |
| Column | | | |
| | Flue Gas | 2,250 | |
| | Air for Combustion | 42,000 | |
| | K₂CO₃ Solution | 65,400 | |
| | Total CO₂ Absorbed (air | 11,800 | |
| | 46%, combustion 54%) | | |
| | Air (ex CO₂) | | 10.4 × 10⁶ |
| | H₂O | | 23,500 |
| | KHCO₃ Slurry | | 53,800 |
| Desorber/Dryer | KHCO₃ Slurry | 53,800 | |
| | Dry K₂CO₃ | | 37,100 |
| | CO₂ & H₂O | | 16,630 |
| Dissolver | Dry K₂CO₃ | 37,100 | |
| | Recycled H₂O | 4,800 | |
| | Added H₂O | 23,500 | |
| | K₂CO₃ Solution | | 65,400 |
| Condenser | CO₂ & H₂O | 16,630 | |
| | CO₂ | | 11,830 |
| | H₂O | | 4,800 |

To operate successfully, the K₂CO₃ must be in solution to form KHCO₃.

The capital costs for this system are as follows. Six pseudo-cooling towers including a spray column cost about $2.0 M. Three 50 KW pumps cost about $3.0 M. A desorber/dryer (rotary kiln) for the reprocessing apparatus costs about $7.6 M. A condenser costs about $8.8 M. A dissolver costs about $9.1 M. The sub-total for these components in therefore about $40.5 M.

The total installed cost based on a Lang Factor of 4 (i.e. that includes construction, engineering, labor, instrumentation, etc) is about $162.0 M. The unit capital equipment costs are based on Ulrick, “A guide to Chemical Engineering Process design and Economics” (1982), updated by using a factor of 2 to bring it up to estimated year 2010 costs.

The estimated operating cost, in US dollars per day, can be determined based on the following costs. The cost from K₂CO₃ loss of 37.3 Ton/Day at a cost of $1200/Ton: $44,800. The heat cost for 29.1×10⁹ BTU/Day of 150 psig steam from a refinery at a cost of $3/MMBTU: 87,300. 20 year deprecation of $162.0 M (162/20×365×0.9): 24,600. Labor, maintenance & other overhead ($1.9 M/yr/365×0.9): 5,800.

These numbers result in a total operating cost per day of $162,500. The total carbon capture and sequestration cost can then be calculated based on the following. The cost per ton Of CO₂ captured (162,500/11,800) is $13.77. The estimated cost of sequestration is $5.00. The total per ton cost for captured and sequestered CO₂ is therefore $18.77. Accordingly, the total per tonne CO₂ captured & sequestered is $20.65, and the total to CCS tonne carbon equivalent is $75.71.

All of the operating and capital costs in this analysis are incremental in nature. The K₂CO₃ price based on a published quote. The steam energy requirement is based on the enthalpy of the reaction. The capital cost only includes 20 yr depreciation. Financing cost and/or estimated profit are not included “Labor, Maintenance & Other” has been estimated as follows: 13 direct labor at $70K/each; 4 maintenance people at $75K each; Supplies etc. (including electricity) $200K. Based on additional calculations, the total steam requirement for this system is 5.9×10⁹+23.2×10⁹=29.1×10⁹ BTLVD.

In addition, it should be noted that similar calculations were carried out for use of this system with natural draft cooling towers. In this case, the assumptions were to the use of a 1000 MW nuclear power plant, cooling towers passing enough air to capture 3900 tons of CO₂ per day, an airflow of 8.33×10⁶ tons per day, a K₂CO₃ circulation of 12,300 tons per day, and K₂CO₃ solution of 21,700 tons per day. Starting from these estimates, it was determined that CO₂ removal of this magnitude could be carried out for a total installation cost of $54.4 million, with daily Operating costs of $57,400, to provide a cost to CCS Tonne Carbon Equivalent of $79.54.

### Example 2

### Plant Configurations Using Waste Heat from Oil Refinery Flare Gas & “Simulated Cooling Towers” to CO₂ Using a Wetted Wall CO₂ Absorber

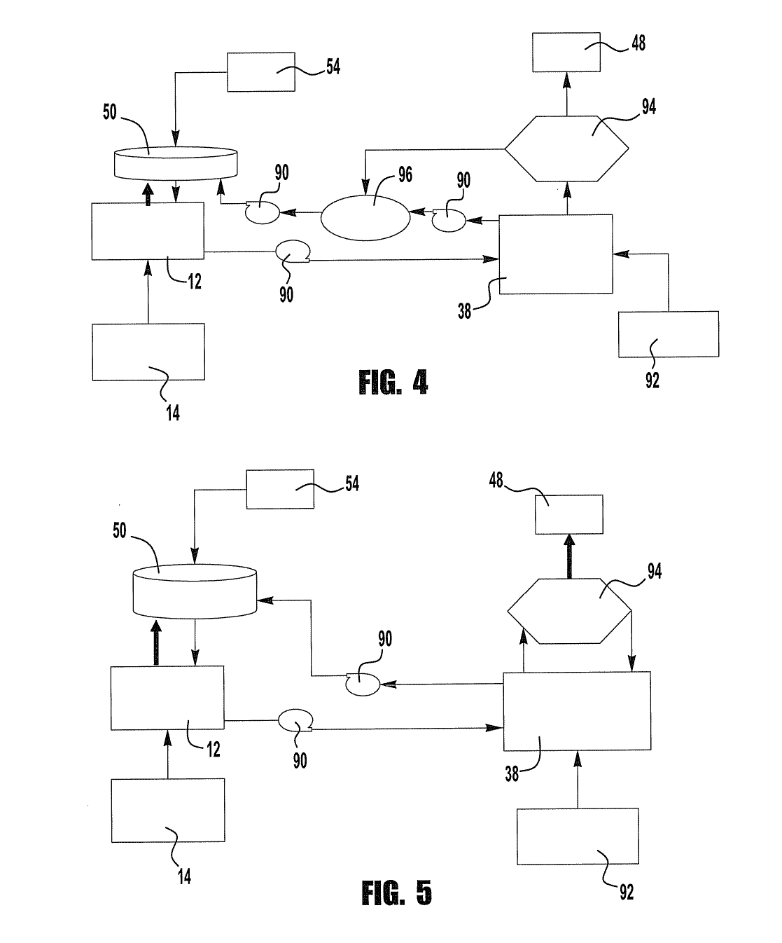

In many respects the second example is similar to that provided in Example 1. A schematic representation of this embodiment of the invention is shown in FIG. 5. Here again waste flue gas from an industrial power source (e.g., a refinery) is used to heat air in a pseudo-cooling tower 50 constructed to simulate the flow of atmospheric air in a power plant cooling tower. The flare gas burners, pointing upward are inserted midway in the pseudo-cooling tower 50 in order to heat the air and combustion gases in the upper section of the pseudo-cooling tower. Therefore the density of the air and combustion gases above this point is lower than that at the bottom of the pseudo-cooling tower causing air flow not unlike that in a power plant natural draft cooling tower with air migrating up in the pseudo-cooling tower.

Assuming a 20° F. (11.1° C.) increase in temperature from the bottom to the top of the pseudo-cooling tower 50, the heat of combustion of the flare gases from a 150,000 bbl/day refinery would provide enough energy to move about 10.4 million ton/day of air and other gases through the pseudo-cooling tower. Moreover since the gas at the top of the pseudo-cooling tower is a mixture of atmospheric air and combustion gas the CO₂ concentration at the top of the pseudo-cooling tower should be about 1277 ppmw.

The CO₂ is absorbed by a K₂CO₃ solution in the CO₂ capture apparatus 12, which in this case is brought into contact with the atmospheric air using a wetted wall process design. Thus, a more dilute carbonate/bicarbonate solution system is used at temperatures ranging from about 40° C. and 90° C., so that these materials are always kept in solution. This approach has the advantage that solids do not have to be handled. On the other hand, additional energy has to be spent heating larger volumes of solution rather than the far more concentrated KHCO₃ precipitate.

The CO₂ is recovered by the thermal decomposition of KHCO₃, carried out in the reprocessing apparatus 38, which also regenerates the K₂CO₃ and water. The recovered CO₂ is then compressed and liquefied in the condenser 94 and pumped to the closest suitable underground geological sequestration sites. Pumps 90 are used to transfer the potassium bicarbonate solution from the CO₂ capture apparatus 12 (i.e., the wetted wall absorber), and the potassium carbonate from the reprocessing apparatus 38 (i.e., the steam heated desorber) to the pseudo-cooling tower 50 where it is used in the CO₂ capture apparatus 12.

The same conditions were used as are described in Example 1.

TABLE 5
Material Balance (in Tons/Day)

| Equipment | Material | Inputs | Outputs |
|---|---|---|---|
| Wetted Wall Absorber | Ambient Air (w 385 ppm | 10.4 × 10⁶ | |
| | CO₂) | | |
| | Flue Gas | 2,250 | |
| | Air for Combustion | 42,000 | |
| | K₂CO₃ Solution (23.8%) | 156,300 | |
| | Total CO₂ Absorbed (air | 11,800 | |
| | 46%, combustion 54%) | | |
| | Air (ex 385 ppm or 3,900 | | 10.4 × 10⁶ |
| | T/D of CO₂) | | |
| | H₂O (water) | | 23,500 |
| | KHCO₃ Sol'n (31.2%) | | 172,700 |
| Direct Steam Desorber | KHCO₃ Sol'n | 172,700 | |
| | H₂O (150 psig steam) | 23,500 | |
| | H₂O | 10,900 | |
| | K₂CO₃ Solution | | 53,900 |
| | H₂O (steam) | | 23,500 |
| | CO₂ & H₂O | | 22,700 |
| Condenser | CO₂ & H₂O | 22,700 | |
| | CO₂ | | 11,800 |
| | H₂O | | 10,900 |

To operate successfully, the K₂CO₃ & KHCO₃ must be kept in solution at all times. The solution is heated with direct steam injection in the direct steam heated desorber (the reprocessing apparatus) from 40° C. to 90° C. in order to generate CO₂. The K₂CO₃ solution is cooled down by the evaporation of the water in the wetted wall absorber, bringing the temperature to 40° C. The solution on the wetted surface is exposed to a crossflow of air that decreases its loss to the high velocity air stream.

The capital cost for this embodiment of the invention is based on the following specific costs. Six absorption wetted wall units cost about $5.4 M. Two 50 KW pumps cost about $1.3 M. A steam heated direct desorber costs about $3.3 M. A condenser costs about $5.2 M. The sub-total based on these components is therefore about $15.2 M.

The total installed cost using a Lang factor of 4 {i.e. that includes construction, engineering, labor, instrumentation, etc) is therefore about $60.8 M. The unit capital equipment costs are based on Ulrick, “A guide to Chemical Engineering Process design and Economics” (1982) updated by using a factor of 2 to bring it up to estimated year 2010 costs.

The estimated operating cost, in US dollars per day, for this embodiment of a CO2 removal system using a wetted wall for absorption of CO₂ is based on the following. The K₂CO₃ loss of 37.3 ton/day at a cost of $1200/Ton: $44,800. The heating cost from 46.9×10⁹ BTU/day 150 psig steam from a refinery at $3/MMBTU: $140,700. A 20 year deprecation of $60.8 M (60.8/20×365×0.9): $9,300. Labor, maintenance & other overhead ($1.6 M/yr/365×0.9): $4,900. This results in a total operating cost per day of $199,700.

The total carbon capture and sequestration cost can then be calculated based on the following. The cost per ton Of CO₂ captured (199,700/11,800) is $16.92. The estimated cost of sequestration is $5.00. The total per ton cost for captured and sequestered CO₂ is therefore $21.92. Accordingly, the total per tonne CO₂ captured & sequestered is $24.11, and the total to CCS tonne carbon equivalent is $88.41. Based on additional calculations, the total steam requirement for this system is 23.3×10⁹+2.1×10⁹+21.5×10⁹=46.9×10⁹ BTLVD.

In addition, it should be noted that similar calculations were carried out for use of this system with natural draft cooling towers. In this case, the assumptions were to the use of a 1000 MW nuclear power plant, cooling towers passing enough air to capture 3900 tons of CO₂ per day, an airflow of 8.33×10⁶ tons per day, and K₂CO₃ solution of 53,200 tons per day. Starting from these estimates, it was determined that CO₂ removal of this magnitude could be carried out for a total installation cost of $25.2 million, with daily operating costs of $69,200, to provide a cost to CCS Tonne Carbon Equivalent of $91.72.

### Example 3

### Flare Gas Required to Move Air from Atmosphere Through Pseudo-Cooling Tower

The amount of air that can be heated by flare gas produced by a 150,000 bbl/day oil refinery is based on the following numbers and calculations. The conditions are those described in Example 1. The CO₂ output from flare gas combustion produced by average refinery is about 0.0425 T CO₂/bbl, based on US Refineries CO₂=304.8×10⁶ T CO₂/yr & Capacity=17.7×10⁶ bbls oil/day). The BTU value of flare gas=1583 BTU/ft³ & CO₂=0.203 lbs/ft³ of flare gas, based on composition & heat of combustion Hydrocarbon Processing tables. Lb CO₂ZMMBTU=0.203×10^{6/1583=128} lb/MMBTU.

The amount Of CO₂ from flare gas at 150,000 bbl/D=150×10³×0.0425=6,375 T CO₂/D. The heat generated by flare gas=6375×2000/128 lb/MMBTU=0.1×10¹² BTU/D. The amount of atmospheric air that can be heated to raise temp by 20° F. is calculated by tons Air/D=0.1×10¹² BTU/D/(29 lb/mol×7 BTU/mol-F 20° F.×2000)=10.4×10⁶ T/D. The CO₂ capture from atmospheric air (assuming 90% recovery) is calculated as follows. (584 T CO₂/10⁶ Air)×10.4×10⁶ T Air×0.90=5,441 T CO₂ZD. Note that the tons of CO₂ in Air is equivalent to 385 ppmv. The combustion of flare gas will create additional CO₂ which can be processed together with that from atmospheric air, allowing the pseudo-towers and the other components of the CO₂ removal system to operate even more efficiently.

### Example 4

### Evaluation and Proposed Design for a CO₂ Removal System Using a Wind Capture Apparatus

To provide some context for evaluating the use of wind to drive a CO₂ removal system, it can be compared to a 1-GW Power Plant Cooling Tower-associated CO₂ removal operation that could process enough air to capture about 3,900 Ton of CO₂/day. Assuming that: (1) wind speed of 6 m/s (or 1.7×10⁶ ftZD), which is the lowest speed that is acceptable for most commercial wind farm operations; (2) cooling towers that pass about 8.33×10⁶ ton of air/day (16.7×10⁹ lb/D or 222.7×10⁹ ft³/D) in order to harvest 3,900 tonnes of CO₂; (3) the density of air at sea level is about 1.2 kg/m³ (1.2 gZL or 0.075 lbZft³); (4) the weight percent of CO₂ in atmospheric air is about 0.0607 (equivalent to 385 ppmv); and (5) a wind capacity factor of 30% of maximum, which is reasonable a number based on the percentage of the time during the course of a year that a good wind is expected to blow, we can determine the size of a comparable wind facility. Depending on its location most operators use a capacity factor of 25% to 40%.

Using these numbers, one can determine that the amount of open space facing the wind that would be equal the CO₂ capture capacity of cooling towers associated with a 1 GW industrial power source would be about 436.7×10³ ft² (222.7×10⁹ ft³/D/(1.7×10⁶ ft/D*0.30). Because this amount of space is probably too large to be contained within a single enclosure, particularly since the enclosure would likely have to be 30 to 45 feet above the ground, it is preferable to divide this space into portions, resulting in, for example, about twenty five 300 ft by 60 ft enclosures.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Having thus described the invention, it is now claimed:

I/We claim:

1. A system for removing carbon dioxide (CO₂) from atmospheric air comprising a cooling tower, a pseudo-cooling tower, or a wind capture device, a CO₂ capture apparatus positioned to contact atmospheric air moving towards the cooling tower or moving within the pseudo-tower or wind capture device, and a reprocessing apparatus in communication with the CO₂ capture apparatus, wherein the CO₂ capture apparatus includes a CO₂ binding agent that binds to CO₂ in atmospheric air, and the reprocessing apparatus releases CO₂ from the binding agent at a temperature of about 200° C. or less, directs the released CO₂ to a CO₂ storage chamber, and returns the binding agent to the CO₂ capture apparatus.

2. The system for removing carbon dioxide of claim 1, wherein, the system comprises a cooling tower that is a natural draft cooling tower.

3. The system for removing carbon dioxide of claim 1, wherein the system comprises a cooling tower that is a mechanical cooling tower.

4. The system for removing carbon dioxide of claim 1, wherein the system comprises a pseudo-cooling tower.

5. The system for removing carbon dioxide of claim 4, wherein the pseudo-cooling tower comprises a flare gas burning apparatus within the upper half of the tower.

6. The system for removing carbon dioxide of claim 1, wherein the CO₂ capture apparatus is-positioned to contact atmospheric air moving towards an air inlet of the cooling tower before the atmospheric air enters the air inlet.

7. The system for removing carbon dioxide of claim 1, wherein the wind capture device comprises one or more apparatus including a first reservoir with open ends facing the wind where CO₂ from the air combines with a binding agent, and then the binding agent flows to a second reservoir where the CO₂ is separated from the binding agent.

8. The system for removing carbon dioxide of claim 1, wherein the system comprises a wind capture device and the system is proximate to a wind turbine that provides energy to operate one or more components of the system.

9. The system for removing carbon dioxide of claim 1, wherein the CO₂ capture apparatus comprises a fluidized bed or a fixed bed reactor.

10. The system for removing carbon dioxide of claim 1, wherein CO₂ capture apparatus comprises a spray tower.

11. The system for removing carbon dioxide of claim 1, wherein the CO₂ capture apparatus comprises a wetted wall apparatus, wherein the CO₂ binding agent is in an aqueous solution and does not precipitate during the capture or release of CO₂.

12. The system for removing carbon dioxide of claim 1, wherein CO₂ binding agent releases bound CO₂ when heated to at least 80° C.

13. The system for removing carbon dioxide of claim 12, wherein the system comprises a cooling tower or a pseudo-cooling tower and the heat to release CO₂ from the CO₂ binding agent is provided by waste heat from a proximate industrial power source.

14. The system for removing carbon dioxide of claim 12, wherein the CO₂ binding agent is an immobilized amine capable of absorbing CO₂ at a first temperature and releasing the CO₂ at a second higher temperature.

15. The system for removing carbon dioxide of claim 12, wherein the CO₂ binding agent is a salt capable of absorbing CO₂ at a first temperature and releasing the CO₂ at a second higher temperature.

16. The system for removing carbon dioxide of claim 15, wherein the salt is potassium carbonate.

17. The system for removing carbon dioxide of claim 15, wherein the salt is sodium carbonate.

18. The system for removing carbon dioxide of claim 1, wherein the CO₂ capture apparatus further comprises a catalyst that improves the kinetics of CO₂ absorption.

19. The system for removing carbon dioxide of claim 18, wherein the catalyst comprises piperazine, carbonic anhydrase enzyme, or a synthetic carbonic anhydrase enzyme analog.

20. The system for removing carbon dioxide of claim 1, wherein the CO₂ binding agent precipitates from an aqueous solution after absorbing CO₂.

21. A system for removing carbon dioxide (CO₂) from flue gas, comprising a CO₂ capture apparatus positioned to contact flue gas moving from or within a smokestack and a reprocessing apparatus in communication with the CO₂ capture apparatus, wherein the CO₂ capture apparatus includes a CO₂ binding agent that binds to CO₂ in atmospheric air, and the reprocessing apparatus releases CO₂ from the binding agent at a temperature of about 200° C. or less, directs the released CO₂ to a CO₂ storage chamber, and returns the binding agent to the CO₂ capture apparatus.

22. The system for removing carbon dioxide of claim 21, wherein the CO₂ binding agent is potassium carbonate.

23. The system for removing carbon dioxide of claim 21, wherein the CO₂ capture apparatus comprises a spray tower.

24. The system for removing carbon dioxide of claim 21, wherein the CO₂ capture apparatus comprises a wetted wall, wherein the CO₂ binding agent is in an aqueous solution and does not precipitate during the capture or release of CO₂.

25. The system for removing carbon dioxide of claim 24, wherein the CO₂ binding agent is potassium carbonate.

26. The system for removing carbon dioxide of claim 25, wherein the potassium carbonate forms potassium bicarbonate upon binding of CO₂, the concentration of the potassium bicarbonate in the aqueous solution is from about 25% to about 35% after absorption of CO₂ at a first temperature, and the concentration of potassium carbonate is from about 15% to about 25% after release of CO₂ at a second higher temperature.

27. The system for removing carbon dioxide of claim 21, wherein the CO₂ binding agent precipitates from an aqueous solution after absorbing CO₂.

28. A method for removing carbon dioxide (CO₂) from atmospheric air comprising the steps of providing a large volume flow of atmospheric air to a CO₂ capture apparatus that includes a CO₂ binding agent, absorbing CO₂ from the large volume flow of atmospheric air to form complexed binding agent, transporting the complexed binding agent to a reprocessing apparatus that releases CO₂ from the complexed binding agent at a temperature of about 200° C. or less to regenerate CO₂ binding agent, removing the released CO₂ from the reprocessing apparatus, and returning the CO₂ binding agent from the reprocessing apparatus to the CO₂ capture apparatus.

29. The method for removing carbon dioxide of claim 28, further comprising the step of storing the CO₂ released from the reprocessing apparatus, using the CO₂ released from the reprocessing apparatus to stimulate growth of photosynthetic organisms such as algae, beverage production, or enhanced oil recovery from underground petroleum reserves.

30. The method for removing carbon dioxide of claim 28, wherein the CO₂ is released from the complexed binding agent by heating the complexed binding agent to at least 80° C.

31. The method for removing carbon dioxide of claim 30, wherein the complexed binding agent is heated by waste heat.

32. The method for removing carbon dioxide of claim 28, wherein the large volume flow of air comprises at least one million tons of air per day.

33. The method for removing carbon dioxide of claim 28, wherein the method removes at least 500 tons of CO₂ from atmospheric air per day.

34. The system for removing carbon dioxide of claim 1, wherein the CO₂ capture apparatus is positioned to contact atmospheric air within the pseudo-tower.
